(19) United States
(12) Patent Application Publication
(10) Pub. No.: US 2015/0005735 A1
(43) Pub. Date: Jan. 1, 2015

(54) Method and Apparatus for Castration of Animals

(71) Applicant: SVW Technologies Pty Ltd, Normanhurst, NSW (AU)

(72) Inventor: Peter St Vincent Welch, Normanhurst (AU)

(21) Appl. No.: 14/370,761

(22) PCT Filed: Jan. 7, 2013
(86) PCT No.: PCT/AU2013/000010

§ 371 Date: Jul. 4, 2014

(30) Foreign Application Priority Data
Jan. 6, 2012 (AU) 2012900049

(51) Int. Cl. A61D 1/06; A61M 5/30

(52) U.S. Cl. CPC A61D 1/06; A61M 5/30
USPC 604/500; 604/68

(57) ABSTRACT

A method and apparatus of applying a chemical treatment solution for effective castration of an animal through inducing cellular necrosis in or about the spermatic cord. The apparatus including a needlefree applicator head comprising a plurality of injector tubes for simultaneous treatment across a target areas. The target area being preferably clamped against the applicator head.

### FIELD OF THE INVENTION

The present invention relates to castration of animals, and in particular to castration of large animals using a chemical treatment.

The invention has been developed primarily for use as a method and apparatus for castration of animals using a non-surgical chemical treatment and will be described hereinafter with reference to this application. However, it will be appreciated that the invention is not limited to this particular field of use.

### BACKGROUND OF THE INVENTION

Any discussion of the prior art throughout the specification should in no way be considered as an admission that such prior art is widely known or forms part of the common general knowledge in the field.

As an example for the use of this technology is the following application. The practice of animal castration (in particular calves and lambs) has traditionally been conducted in Australia using a either a knife to remove the testicles, mechanical methods to crush the vas deferens and associated blood vessels or the use of a scrotal rubber ring. Castration of animals has traditionally been performed without an anaesthetic. Castration has been painful to the animal and alternative procedures are being investigated to reduce the pain associated with the practice.

### OBJECT OF THE INVENTION

It is an object of the present invention to overcome or ameliorate at least one of the disadvantages of the prior art, or to provide a useful alternative.

It is an object of the invention in its preferred form to provide a method and apparatus of applying a chemical treatment for effective castration of an animal through inducing cellular necrosis in the spermatic cord.

### SUMMARY OF THE INVENTION

According to the invention there is provided an improved method to castrate animals by inducing cellular necrosis in the spermatic cord including the vas deferens, blood vessels and neural network to effectively castrate the animal. Preferably, this method provides a relative reduction in levels of stress suffered by the animal.

According to the invention there is provided an applicator apparatus for effective castration of an animal, the apparatus including:
a body having an applicator head;
wherein, the applicator head can be located over a target area, such that with pressure applied by the applicator head toward the target area, the applicator head treats the target area for effective castration of the animal.

According to the invention there is provided an applicator apparatus for effective castration of an animal, the apparatus including:
a body having a clamping assembly;
the clamping assembly including a clamp element and an applicator head;
the clamp element opposes the applicator head, such that respective movement brings the clamp element and the opposing applicator head from an open configuration to a clamped configuration for clamping a target treatment area between the clamp element and the applicator head;
wherein, in the clamped configuration, the applicator head treats the target area for effective castration of the animal.

Preferably the applicator head is a needlefree applicator head. More preferably, the applicator head comprises a plurality of needlefree injector tubes. Most preferably the plurality of injector tubes protrudes (or extends) a predetermined distance.

Preferably, upon the applicator head being applied over a target area and pressed against the animal, such that the pressure applied by the target area against the applicator head reaches a predetermined pressure, causes a pressure sensitive actuator to deliver (or release or propel) a dose of solution through the applicator head to the target area. More preferably, the pressure sensitive actuator is automatically reset upon release of the applied pressure. Most preferably, the pressure sensitive actuator only enables delivery of a single predetermined dose.

Preferably the clamping assembly enables relative movement of the clamp element towards the applicator head for providing clamping engagement of a target treatment area there between. More preferably, upon the applicator head (or needlefree applicator tubes) being clamped against target area, such that the pressure applied by the target area against the applicator head reaches a predetermined pressure, causes a pressure sensitive actuator to deliver (or release or propel) a dose of solution through the applicator head to the target area. More preferably, the pressure sensitive actuator is automatically reset upon release of the clamping pressure. Most preferably, the pressure sensitive actuator only provides a single predetermined dose.

According to the invention there is provided a method of effective castration of an animal, the method including the steps of:
(a) locating an applicator apparatus such that the spermatic cords traverse through the target treatment area defined by the applicator head;
(b) bringing the applicator head into abutting engagement with the target treatment area;
(c) delivering of a dose of treatment solution at a predetermined pressure.

According to the invention there is provided a method of effective castration of an animal, the method including the steps of:
(a) locating an applicator apparatus with the scrotum between the clamp element and applicator head such that the spermatic cords traverse through the target treatment area;
(b) bringing the clamp element and applicator head into clamping engagement (for example using a relatively movable grip portion or handle or trigger);
(c) delivering of a dose of treatment solution at a predetermined pressure.

Preferably, the applicator apparatus is according to any embodiment applicator apparatus disclosed herein for effective castration of an animal.

Preferably, the treatment solution is according to any embodiment treatment solution disclosed herein for effective castration of an animal.

According to an aspect of the invention there is provided a non-surgical method for the effective castration of an animal. The effective castration of an animal is preferably through causing any one or more of: necrosis, loss of tissue structure, coagulation of tissues, and/or dissolution of anatomical structures about a target treatment area. More preferably, the target treatment area is the spermatic cords.

Preferably, the method comprises administration to the target treatment area an effective amount of a treatment solution capable of causing necrosis. More preferably, the treatment solution is an anionic surfactant. Most preferably, the anionic surfactant is selected from the group consisting of an alkyl sulphate, alkyl benzene sulphonate, and alkylether sulphate.

Preferably, the method comprises administration to the target treatment area an effective amount of a treatment solution capable of causing necrosis. More preferably, the treatment solution is an anionic surfactant in the form of an alkyl sulphate. More preferably, the alkyl sulphate is sodium tetradecyl sulphate or analogs and derivatives thereof.

Preferably, the method comprises administration to the target treatment area an effective amount of a treatment solution capable of causing necrosis. More preferably, the treatment solution is an anionic surfactant in the form of any one or more of the following: sodium decyl sulphate, sodium lauryl sulphate, sodium myristyl sulphate, or sodium cetyl sulphate. Most preferably, the treatment solution includes sodium lauryl sulphate.

### BRIEF DESCRIPTION OF THE DRAWINGS

A preferred embodiment of the invention will now be described, by way of example only, with reference to the accompanying drawings in which:

FIG. 1 is a perspective view of an embodiment apparatus for effective castration of animals according to the invention;

FIG. 2 is a perspective view of the embodiment apparatus of FIG. 1;

FIG. 3 is a front elevation view of the apparatus of FIG. 1;

FIG. 4 is a plan view of the apparatus of FIG. 1;

FIG. 5 is an inline for assembly view of an embodiment replaceable applicator head; and

FIG. 6 is a flow chart of an embodiment method of effective animal castration according to the invention.

### PREFERRED EMBODIMENT OF THE INVENTION

Preferred embodiments relates to a chemical method of effectively castrating animals. The method and apparatus disclosed herein have been developed as a method to reduce animal stress during and following castration, and as an alternative to mechanical castration.

In an embodiment, a sclerosing solution is injected by physical, mechanical or pneumatic means into the scrotum of the animal. The sclerosing solutions can be injected using a needfree injector, for example using a pneumatic means.

It will be appreciated that, targeted injection of sclerosing solution into the scrotum of the animal will cause protein denaturation resulting in damage to the spermatic cord, vas deferens, neural network, veins and capillaries. This damage induces the cellular destruction in the targeted regions including the neural network resulting in necrosis of the targeted tissues.

It will be further appreciated that volume and concentration of the sclerosing agent in the target region will determine the degree of damage to that region. An optimal concentration of the sclerosant can cause irreversible damage to the treated or targeted tissue while leaving adjacent tissue relatively unaffected.

Sclerosing solutions include a class of drugs known as fatty acids or alcohols, or as detergents. Sclerosants work by a mechanism known as protein theft denaturation, in which an aggregation of detergent molecules forms a lipid bilayer in the form of a sheet, a cylinder or a micelle, which disrupts the cell surface membrane. The loss of these essential surface proteins causes a delayed cell death.

Detergent sclerosants (or sclerosing solutions) do not cause haemolysis, or provoke direct intravascular coagulation. The activity of detergent sclerosing solutions is concentration dependent.

When the concentration detergent sclerosant molecules in solution reaches a threshold (known as critical micellar concentration or CMC) nearly all further detergent molecules added to the solution will enter into micelles. Micelles can cause protein theft denaturation, while individual detergent molecules have no toxicity to the vascular endothelium. Accordingly, for each detergent sclerosant, there is a threshold concentration below which the agent causes no (or only minimal) damage or injury to tissue it is applied. This physical property means that detergent sclerosant agents can offer significant benefits over other agents, because they are potent agents that have a predetermined concentration threshold below which they do not close injurious effect on vascular endothelium.

Sclerosing solutions can also include sodium lauryl sulphate (SLS). Sodium lauryl sulphate is an anionic surfactant, which is an analogue of sodium tetradecyl sulphate. This sclerosing agent, when injected into skin tissue, causes necrosis of skin tissue including the sensory nerves. The damage caused to the sensory nerves results in reduced pain within a 1 minute period after the intradermal injection of SLS.

In an embodiment, injection of an anionic surfactant including alkyl sulphate (for example sodium lauryl sulphate or its analogues) at a concentration greater than the critical micellar concentration by physical, mechanical, hydraulic or pneumatic, needle or needlefree means into the scrotum of animals such as sheep and cattle cause damage to the testicular blood vessels and/or vas deferens resulting in effective castration. This injection, particularly when using a needlefree injector, can cause a relatively reduced stress response in the animal when compared to conventional castration methods.

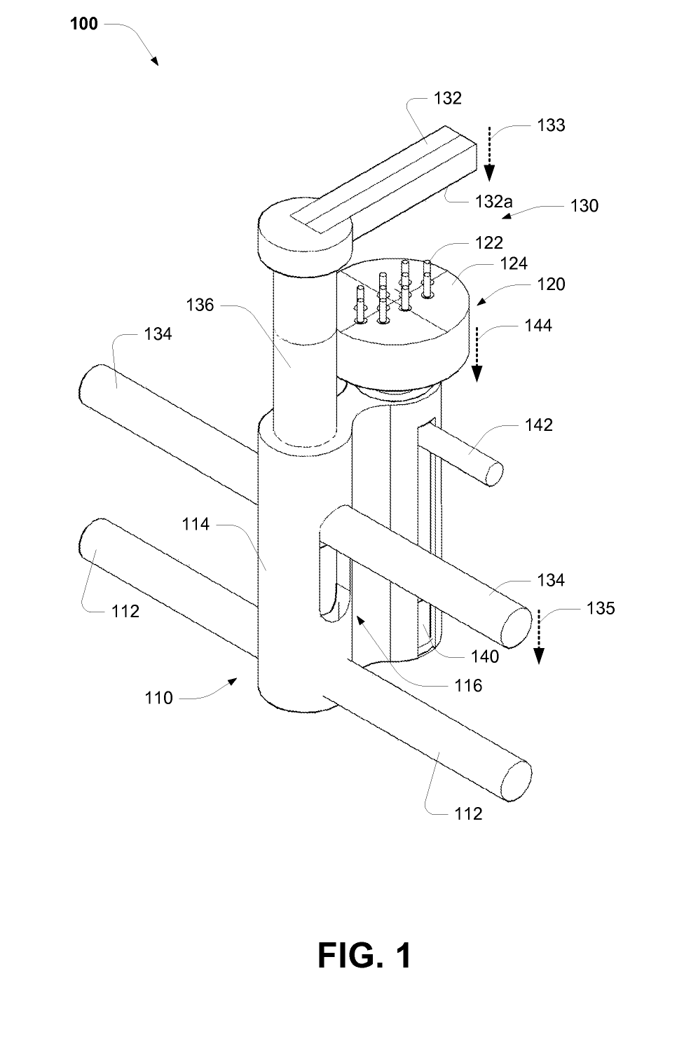

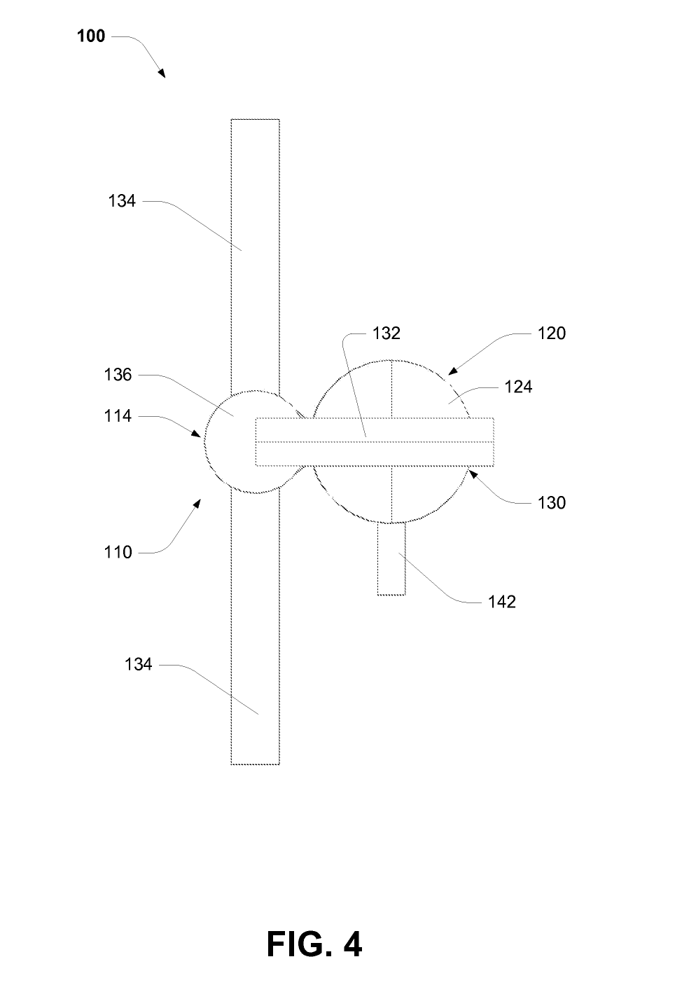

It will be appreciated that the disclosed applicator apparatus shown in FIG. 1 though FIG. 4 is suitable for animals such as cattle and sheep. For some other animals, such as pigs, it is difficult to clamp the scrotum above each testicle to apply a treatment solutions to an appropriate target area for effective castration.

For such animals, an applicator head can be applied directly against the animal body about a target area (such as the spermatic cord) to applying a treatment solution (or sclerosing solution). Accordingly, an embodiment applicator apparatus can have a releasable (or removable) clamping element, such that with the clamping element the applicator head can be directly applied against the animal.

It will be appreciated that the embodiment method and apparatus are not limited to this particular fields of use and could be used in the broader field of hydraulic (no needle) injection of liquids into and through animal skins for mulesing, tail docking and horn tip removal.

An applicator apparatus, by way of example only, as a needlefree injector that can include a manually reset “pressure on demand” element that keeps suspended products in solution until the device is used to administer the injection. An operator or user pressurizes the element, for example by pressing a button on the amplifier, handpiece or remote component. The treatment device typically uses compressed gas (for example carbon-dioxide gas or nitrogen gas) for pressurisation of the system.

An applicator apparatus, by way of example, can be powered by compressed air, carbon-dioxide gas, nitrogen gas or liquefied petroleum gas. A variable pressure pneumatic amplifier, can enable the apparatus to deliver or propel a variable dose of medication directly through the skin and into the subcutaneous or intramuscular layer of tissue. An applicator apparatus can be actuated by contact pressure applied between the animal (or animal skin) and the needlefree head—usually by pressing or holding the applicator head against the animal. A pneumatic amplifier is used to increase the pneumatic pressure of the input gas to a higher hydraulic pressure of the liquid to be injected into the animal. The pneumatic input pressure is regulated with a pressure relief valve. A gas pressure amplifier and pressure regulator can enables the amplifier to converts the gas pressure into hydraulic pressure to the applicator handpiece. The hydraulic pressure can be adjusted to accommodate different animals or application (or treatment) depth.

It will be appreciated that needlefree injectors can be used in animal health applications, for transdermal delivery of vaccines and pharmaceuticals. The needle-free injectors can provide a measured dose range. By way of example only, a dose range can be 0.5 ml to 2.5 ml or 1.0 ml to 5.0 ml.

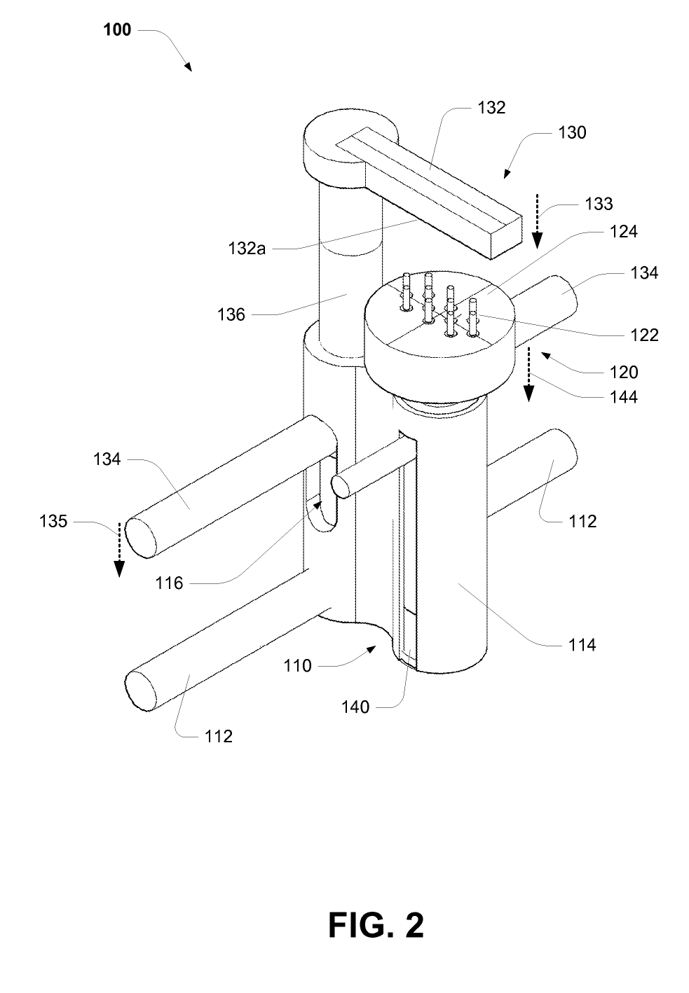

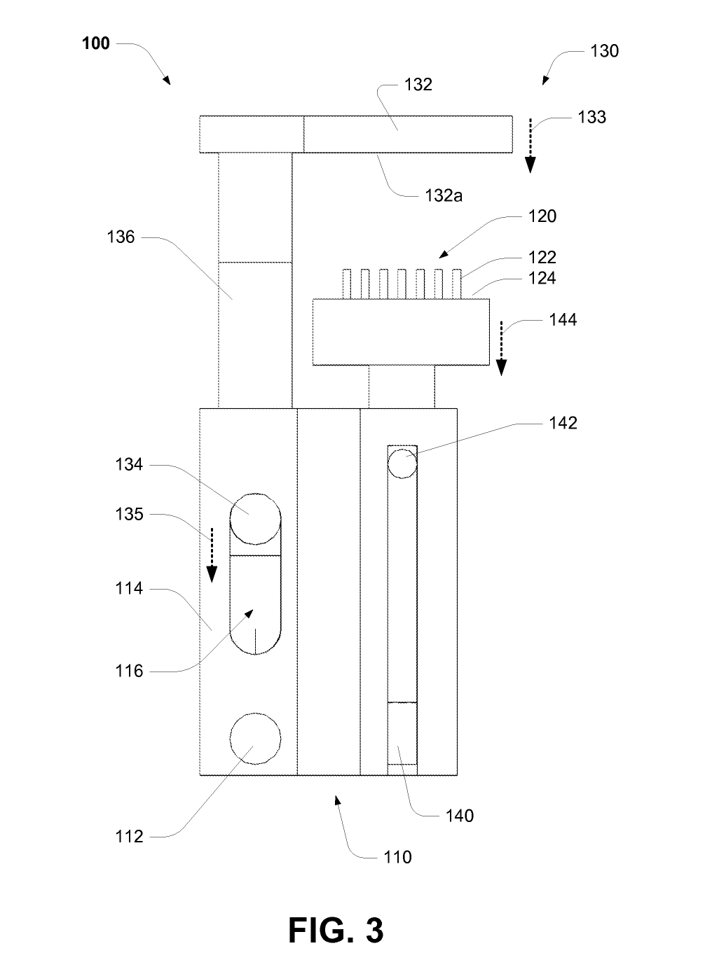

FIG. 1 through FIG. 4 show, by way of example only, an embodiment applicator apparatus 100 for effective castration of an animal.

The embodiment applicator apparatus 100 comprises:
a body 110 having an applicator head 120;
wherein, the applicator head can be located over a target area, such that with pressure applied by the applicator head against or toward the target area, the applicator head treats the target area for effective castration of the animal.

The embodiment applicator apparatus 100 comprises:
a body 110 having a clamping assembly 130;
the claiming assembly including a clamp element 132 and the applicator head 120;
the clamp element 132 opposes the applicator head 120, such that respective movement 133 brings the clamp element and the opposing applicator head from an open configuration to a clamped configuration for damping a target treatment area (not shown) between the clamp element and the applicator head;
wherein, in the clamped configuration, the applicator head treats the target area for effective castration of the animal.

In an embodiment, the applicator head is a needlefree applicator head 120 including a plurality of needlefree injector tubes 122. The plurality of injector tubes protrude (or extend) a predetermined distance from a base element 124.

In an embodiment, upon the applicator head 120 being applied over a target area and pressed against the animal, such that the pressure applied by the target area against the applicator head reaches a predetermined pressure, causes a pressure sensitive actuator to deliver (or release or propel) a dose of solution through the applicator head to the target area. The pressure sensitive actuator automatically delivers a single predetermined dose and resets upon release of the applied pressure.

In an embodiment, upon the clamping assembly 130 enables relative movement of the clamp element 132 towards the applicator head 120 for providing damping engagement of a target treatment area there between. The applicator head 120 (or needlefree applicator tubes 122) are pressed or held against the target area, such that the pressure applied by the target area against the applicator head reaches a predetermined pressure, which causes a pressure sensitive actuator to deliver (or release or propel) a dose of solution through the applicator head to the target area. The pressure sensitive actuator typically provides a single predetermined dose and automatically resets upon release of the clamping pressure.

By way of example, the body no includes a handle (or handles) 112 and a trigger (or triggers) 134 that is moveable with respect to the handle for bringing the support element 132 into clamping engagement with the applicator head 120 about a target treatment area. In this example, the body includes a sleeve portion 114 that enables slidable engagement with a shaft 136 that is rigidly coupled to the clamp element 132. The shaft (or the clamp element) is biased to an open configuration via a biasing element (for example, a spring located within the sleeve portion. The trigger 134 is coupled to the shaft 136 such that movement 135 of the trigger towards the handle causes the shaft to descend into the sleeve portion to thereby bring the clamp element into clamping engagement with the applicator head. In this example, the trigger is guided by an aperture 116 defined by the sleeve portion 114.

It will be appreciated that a handle 112 and trigger 134 can be replicated on either side of the apparatus such that the device can be readily used in left handed and right handed operation and for enabling ease of use after rotation of the device during use on the left side and right side of the animal and enable left handed and right handed operation.

In an embodiment, sensitivity of the pressure sensitive actuator 140 can be in the form of a variable pressure pneumatic amplifier for delivering a single dose application of a chemical provided through an ingress tube 142. In the applicator head, upon application of pressure, can descend 144 into the body for actuating the pressure sensitive actuator.

In an embodiment, the applicator head 120 comprises an array needlefree injector tubes 122 that protrude from a base element 124. The array is configured to enable simultaneous treatment across a target area. The clamp element 132 provides a clamp surface 132a that is located opposite the injector tubes such that a relatively constant pressure can be applied across the target treatment area to each of the needlefree injector tubes.

It will be appreciated that the plurality of injector tubes can be configured (number, spacing and arrangement) to define the treatable area. The applicator head can be therefore be releasably engagable from the body for enabling alternative injector tubes configurations to be used for treating alternative target areas or alternative treatment methods.

In an embodiment, needlefree injection of sclerosing solutions by physical, mechanical or pneumatic means is applied though an applicator head. Typically the applicator head has a plurality of injector egress apertures (each typically defined by an injector tube). The plurality of injector egress apertures enable simultaneous treatment across a target treatment area.

This invention enables the injection of the sclerosing solutions agents though the skin, in particular, through the scrotum of an animal into to the spermatic cords.

By way of example only, the current embodiment uses a detachable replaceable, interchangeable injection plate with an array of small injector tubes. By way of example only, the tubes are stainless steel with an outer diameter of 0.5-1.00 mm and an inner diameter of 0.15 to 0.5 mm (defining an egress aperture). In a preferred embodiments, the outer diameter is 0.95 mm and the inner diameter is 0.25 mm.

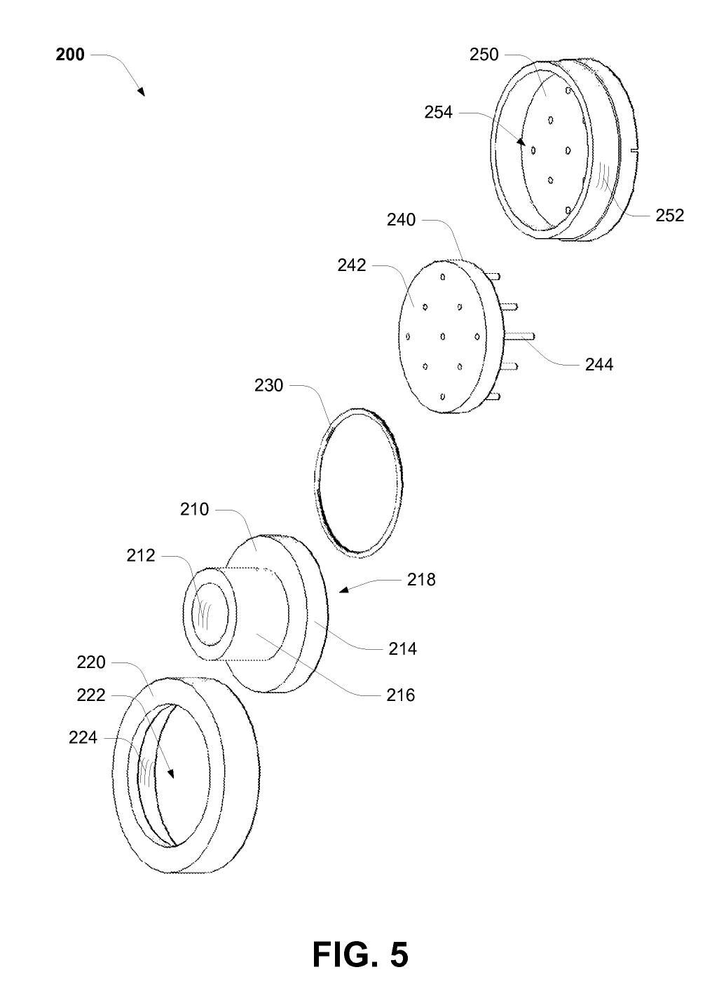

Referring to FIG. 5, an embodiment replaceable applicator head 200 is attachable to an outlet the body not shown, preferably by means of a coupling element 210 having an internally threaded coupling 200 having a central threaded bore or opening 212 surrounded by a flange 214. A coupling nut 220 is interposed between the coupling 200 and body and has a central opening 222 through which passes the generally cylindrical portion 216 of the coupling element 210. A seal such as an O ring 230, is interposed between the front face 218 of the coupling 210 and a rear face 242 of an applicator insert 240. The insert 240 comprises an array of injector tubes 244 and is held in position by a retainer element 250. The retainer element 250 has an externally threaded rim 252 that is received by the internally threaded rim 224 of the retainer nut 220. The retainer element 250 further comprises an array of through openings 254 that correspond with and cooperate with the array of injector tubes 244. The array of injector tubes 244 passes through the array of openings 254 so that threading the nut 220 onto the retainer element 250 creates a clamping force on the insert 240 that compresses the seal 230 and thus creates a void between the back face 242 of the insert and the front face 218 of the coupling.

Pressurised fluid in this void is dischargeable through the injector tubes 244. In some embodiments, the injector tubes 244 have sufficient length to protrude from the retainer element 250.

In an embodiment, the applicator insert 240 comprises an interchangeable, plastic disc or body through which pass an array of needlefree injector tubes. The injector tubes are sealed with respect to the insert body so that pressurised fluids adjacent to the back face can only exit through the small through holes or bores that pass through each of the needlefree injector tubes.

By way of example, an injector insert can be manufactured by inserting the stainless steel injector tubes into, or moulding a polymeric body around the injector tubes. In either case, it is preferred that the injectors tubes be flush with respect to the back side of the insert. The tip if each injector tube can be generally flat and perpendicular to the long axis of the injector; generally flat but has a rounded or radiused peripheral edge; entirely radiused or domed.

The injector tubes can be arranged in a number of variable array configurations including linear, square, rectangular, circular or combinations of these, with a range of distances between the tubes. In a preferred embodiment the tubes are arranged in a rectangular array to enable a rectangular treatment area.

By way of example, the array of injector tubes may be individually secured to the applicator by variety of means. The array of tubes can be carried by a plastic disc or metal disc (such as stainless steel). The advantage of the plastic disc is that it may be manufactured by plastic injection with the tubes encompassed in the disc during the manufacturing process. The replaceable disc allows for the easy replacement of the tubes (as an array) if they become blocked or damaged in any way. The replaceability of the injector insert facilitates the use of different arrays for different situations. A new retainer element may be required to accommodate some arrays.

It will be appreciated that, when the injector tubes are pressed against the skin of an animal, they tend to compress the skin at and around the point of contact.

In use, an embodiment applicator apparatus can provide effective castration of animals.

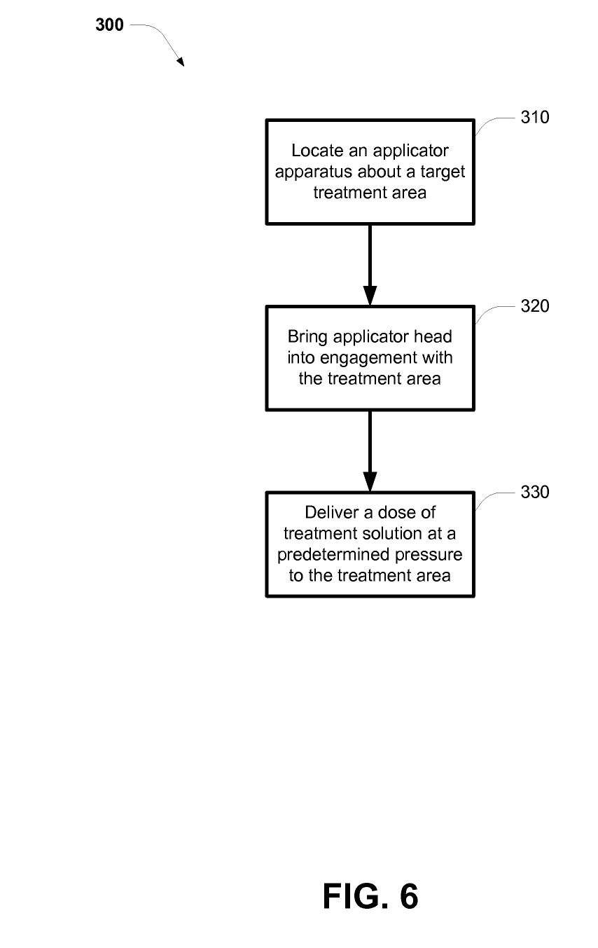

By way of example only, referring to FIG. 6, an embodiment method 300 of providing effective castration of animal can include the following steps:
STEP 310 locating an applicator apparatus such that the spermatic cords traverse through the target treatment area defined by the applicator head;
STEP 320 bringing the applicator head into abutting engagement with the target treatment area;
STEP 330 delivering of a dose of treatment solution at a predetermined pressure.

By way of example only, referring to FIG. 6, an embodiment method 300 of providing effective castration of animal can include the following steps:
STEP 310 locating an applicator apparatus with the scrotum between the damp element and applicator head such that the spermatic cords traverse through the target treatment area;
STEP 320 bringing the clamp element and applicator head into clamping engagement (for example using the trigger);
STEP 330 delivering of a dose of treatment solution at a predetermined pressure.

It will be appreciated that, by applying pressure to the applicator head can enable an automatic delivery of a dose of treatment solution at a predetermined pressure.

In an embodiment, the treatment solution is needlefreely injected to the target area (for example, the spermatic cords) for causing effective castration of the animal.

In an embodiment, the treatment solution is a sclerosing solutions, which includes sodium lauryl sulphate (SLS). Needlefree injection of the treatment solution to the target area can cause effective castration of the animal. For example, needlefree injection of a sclerosing solutions (such as sodium lauryl sulphate) to the target area (such as the spermatic cords) can cause effective castration of the animal through necrosis of the target area.

It will be appreciated that the predetermined pressure and dose volume will be dependent on many factors including the animal being treated, and the location and size of the target area. A plurality of dose shots can be applied to the same target area. The dosage pattern applied to a target area is defined by the configuration of needlefree injector tubes on the applicator head. The number and configuration of the needlefree injector tubes can be dependent on the intended use. Further, each applicator head can be releasable coupled to the body for enabling selection of an alternative applicator head (or configuration of the needlefree injector tubes).

It will be appreciated that a range of applicator heads (each with a different array of plurality of needlefree injector tubes and array configuration) can be provided for effective castration of different animals.

The applicator apparatus or treatment solution can be further adapted to provide delivery of a local anaesthetic to or about the treatment area.

By way of example only, in a first field trial, a calf was treated using an apparatus being similar to that disclosed in FIG. 1 with a treatment solution of sodium lauryl sulphate (SLS). In this first trial:
the target area including the left spermatic cord was delivered a single dose of 2.5 mL across an applicator head having 8 needlefree injector tubes under pneumatic pressure of 95 psi (hydraulic pressure of liquid approximately 9500 psi);
the target area including the right spermatic cord was delivered two dose each of 2.5 mL dose across an applicator head having 8 needless injector tubes under pressure of 95 psi;
the calf was sedated then placed in lateral recumbancy when administering the treatment solution, such that the applicator was held on the anterior aspect of the neck of the scrotum when administering the treatment solution;
upon examination, after only a few minutes, sufficient penetration of the treatment solution within the spermatic cords was observed—with loss of tissue structure and coagulation of tissues.

By way of example, in a second field trial, a calf was treated using an apparatus being similar to that disclosed in FIG. 1 with a treatment solution of sodium lauryl sulphate (SLS). In this second trial and applicator head having 10 needless injector tubes was used to observe impact of a lower delivery momentum for the treatment solution:
the target area including the left spermatic cord was delivered a single dose of 2.5 mL across an applicator head having 10 needless injector tubes under pressure of 95 psi;
the target area including the right spermatic cord was delivered a single dose of 2.5 mL across an applicator head having 10 needless injector tubes under pressure of 95 psi;
the calf was sedated then placed in lateral recumbancy when administering the treatment solution, such that the applicator was held on the anterior aspect of the neck of the scrotum, spermatic cords were further pinched against the lateral side of the neck of the scrotum to provide better control over position of the cords during administration of the treatment solution;
upon examination, after 10 minutes from administering the treatment solution, sufficient penetration of the treatment solution within the spermatic cords was observed—with dissolution of anatomical structures within the spermatic cords.

As sodium lauryl sulphate (SLS) can diffuse through tissues for several minutes after administration, causing additional damage to anatomical structures.

The configuration of the applicator head (for example number and arrangement of injector tubes), pressure settings and dose volume can be configure to provide suitable penetration of SLS and tissue disruption that is anticipated to be required to disrupt nerve and blood supplies to the testes for effective castration of an animal.

It has been observed that Lignocaine (containing lignocaine hydrochloride 20 mg/mL and benzyl alcohol 10 mL/L as preservative) failed to penetrate beyond dermal structures for similar trial configuration to those outlined above. Failure of lignocaine to penetrate to spermatic cords at these same settings is in accordance with previous experience of transcutaneous injection of saline and SLS into sheep skin. Accordingly, the applicator configuration and settings to be tailored to the physical characteristics of the treatment solution being delivered, such that the required depth of penetration across the treatment area is achieved.

In the above trials, green food dye containing tartrazine (food colour 102) and brilliant blue (food colour 133) to 21%, citric acid (food acid 330) and sodium benzoate (food preservative 211) was added to 10% v/v to aid observation of the distribution of the injected solution.

It will be appreciated that, in an attempt to reduce splattering of drug on the skin surface and to increase the depth of penetration, the applicator can be modified to increase the delivery pressure of the treatment solution and to increase the pressure between the injector tubes and scrotal tissue at the time of drug delivery.

It will be appreciated that the animal can be sedated and placement in lateral recumbancy for administration of the treatment solution from the anterior aspect of the scrotum with spermatic cords pulled to the lateral wall of the neck of the scrotum for improved operator control during application.

In an alternative configuration, by way of example, the animal can be standing in a race with the animal restrained by the neck in a suitable cattle crush, The applicator is then applied to the scrotum with the animal standing so that the scrotum is hanging and thereby reducing the manipulation of the applicator prior to the injection necrosing agent or local anaesthetic into the scrotum.

It will be appreciated that the applicator and/or treatment solution can be adapted to further deliver a local anaesthetic to spermatic cords.

It will be appreciated that the illustrated embodiments disclose a method and apparatus of applying a chemical treatment for effective castration of an animal through inducing cellular necrosis in the spermatic cord.

It will be appreciated that the disclosed embodiments disclose a method and apparatus for effective castration of animals by inducing cellular necrosis in the spermatic cord including the vas deferens, blood vessels and neural network to effectively castrate the animal. This method can also provide a relative reduction in levels of stress suffered by the animal.

Although the invention has been described with reference to specific examples, it will be appreciated by those skilled in the art that the invention may be embodied in many other forms.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

Similarly, it is to be noticed that the term coupled, when used in the claims, should not be interpreted as being limitative to direct connections only. The terms “coupled” and “connected”, along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical, or that two or more elements are not in direct contact with each other but yet still co-operate or interact with each other.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

As used herein, unless otherwise specified the use of terms “horizontal”, “vertical”, “left”, “right”, “up” and “down”, as well as adjectival and adverbial derivatives thereof (e.g., “horizontally”, “rightwardly”, “upwardly”, etc.), simply refer to the orientation of the illustrated structure as the particular drawing figure faces the reader, or with reference to the orientation of the structure during nominal use, as appropriate. Similarly, the terms “inwardly” and “outwardly” generally refer to the orientation of a surface relative to its axis of elongation, or axis of rotation, as appropriate.

Similarly it should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

It will be appreciated that an embodiment of the invention can consist essentially of features disclosed herein. Alternatively, an embodiment of the invention can consist of features disclosed herein. The invention illustratively disclosed herein suitably may be practiced in the absence of any element which is not specifically disclosed herein.

1. An applicator apparatus for effective castration of an animal, the apparatus including:
a body having an applicator head;
wherein, the applicator head can be located over a target area, such that with pressure applied by the applicator head toward the target area, the applicator head treats the target area with a treatment solution for effective castration of the animal.

2. The applicator apparatus according to claim 1, wherein the applicator head is a needlefree applicator head.

3. The applicator apparatus according to claim 1, wherein the applicator head comprises a plurality of injector tubes.

4. The applicator apparatus according to claim 3, wherein the plurality of injector tubes each protrude a predetermined distance.

5. The applicator apparatus according to claim 1, wherein, upon the applicator head being applied over a target area and pressed against the animal, such that the pressure applied by the target area against the applicator head reaches a predetermined pressure, a pressure sensitive actuator deliver a dose of treatment solution through the applicator head to the target area.

6. The applicator apparatus according to claim 5, wherein the pressure sensitive actuator is automatically reset upon release of the applied pressure.

7. The applicator apparatus according to claim 1, the apparatus further including:
a clamping assembly;
the clamping assembly including a clamp element and the applicator head;
the clamp element opposes the applicator head, such that respective movement brings the clamp element and the opposing applicator head from an open configuration to a clamped configuration for clamping a target treatment area between the clamp element and the applicator head;
wherein, in the clamped configuration, the applicator head treats the target area for effective castration of the animal.

8. The applicator apparatus according to claim 7, wherein the clamping assembly enables relative movement of the clamp element towards the applicator head for providing clamping engagement of a target treatment area there between; such that the pressure applied by the target area against the applicator head reaches a predetermined pressure for causing a pressure sensitive actuator to deliver a dose of solution through the applicator head to the target area.

9. The applicator apparatus according to claim 7, wherein the target area includes a spermatic cord of the animal.

10. The applicator apparatus according to claim 1, wherein the treatment solution includes an effective amount of an anionic surfactant.

11. The applicator apparatus according to claim 10 wherein the anionic surfactant is an alkyl sulphate.

12. The applicator apparatus according to claim 10, wherein anionic surfactant is selected from any one or more of the set comprising: sodium decyl sulphate, sodium lauryl sulphate, sodium myristyl sulphate, or sodium cetyl sulphate.

13. The applicator apparatus according to claim 10, wherein anionic surfactant is sodium lauryl sulphate.

14. The applicator apparatus according to claim 1, wherein castration of the animal is effected by the treatment solution causing necrosis of or about a spermatic cord of the animal.

15. A method of effective castration of an animal, the method including the steps of:
(a) locating an applicator apparatus such that the spermatic cords traverse through a target treatment area defined by the applicator head;
(b) bringing the applicator head into abutting engagement with the target treatment area; and
(c) delivering of a dose of treatment solution at a predetermined pressure into the target area.

16. The method according to claim 15, wherein
the applicator apparatus is located with the scrotum between a clamp element and applicator head, such that the spermatic cords traverse through a target treatment area defined by the applicator head; the method further comprising the step of: bringing the clamp element and applicator head into clamping engagement about the target area.

17. The method according to claim 16, wherein the treatment solution delivered into the target treatment area is an effective amount of treatment solution capable for causing necrosis about the target area.

18. The method according to claim 16, wherein the target treatment area is the spermatic cords.

19. The method according to claim 16, wherein treatment solution includes an effective amount of an anionic surfactant.

20. The method according to claim 16, wherein treatment solution includes an effective amount of sodium lauryl sulphate.

21. (canceled)
